(19) United States
(12) Patent Application Publication
(10) Pub. No.: US 2025/0092060 A1
(43) Pub. Date: Mar. 20, 2025

(54) PHENOLIC COMPOUNDS SUBSTITUTED WITH NON-RADIOACTIVE ISOTOPES AND USES THEREOF

(71) Applicant: SEQUL NATIONAL UNIVERSITY R&DB FOUNDATION, Seaoul (KR)

(72) Inventors: Hyun-Woo RHEE, Seoul (KR); Myeong-Gyun KANG, Seoul (KR)

(73) Assignee: SEQUL NATIONAL UNIVERSITY R&DB FOUNDATON, Seaoul (KR)

(21) Appl. No.: 18/572,979

(22) PCT Filed: Aug. 29, 2023
(86) PCT No.: PCT/KR2023/012759

§ 371 Date: Dec. 21, 2023

(30) Foreign Application Priority Data
Aug. 29, 2022 (KR) 10-2022-0108530

(51) Int. Cl. C07D 495/04; C07D 233/02

(52) U.S. Cl. CPC C07D 495/04; C07D 233/02

(57) ABSTRACT

The present invention relates to phenolic compounds substituted with non-radioactive isotopes and the uses thereof, and more specifically to phenolic compounds in which some elements of biotin-phenol or desthiobiotin-phenol are substituted with non-radioactive isotopes and the uses thereof as probes for APEX family enzymes used in proximity molecular labeling. According to the present invention, proteins that are present in spaces that are not separated by membranes (e.g., mitochondrial cristae lumen), which previously could not be analyzed, can be identified, and the quantitative comparative analysis of protein expression in cells in different environments is possible, and it has the advantage of being able to quantitatively compare and analyze protein ratios in different adjacent spaces, and particularly, it has the advantage of being able to label proteins economically compared to the conventional technique of labeling proteins using heavy-carbon labeled amino acids.

### CROSS-REFERENCE TO RELATED APPLICATION

This application claims priority to and the benefit of Korean Patent Application No. 10-2022-0108530, filed on Aug. 29, 2022, the disclosure of which is incorporated herein by reference in its entirety.

### TECHNICAL FIELD

The present invention relates to phenolic compounds substituted with non-radioactive isotopes and the uses thereof, and more specifically to phenolic compounds in which some elements of biotin-phenol or desthiobiotin-phenol are substituted with non-radioactive isotopes and the uses thereof as probes for APEX family enzymes used in proximity molecular labeling.

### BACKGROUND ART

It has been revealed through imaging using an electron microscope that the 20 intermembrane space of mitochondria is structurally divided into a peripheral space and a cristae lumen, and it has also been revealed through cryogenic electron microscopy that the oxidative phosphorylation complex that constitutes the electron transport system exists in the cristae lumen.

As such, it can be expected that mitochondrial functions in these two structurally distinct spaces (i.e., peripheral space and cristae lumen) will be different from each other, but since the two spaces are not separated by a membrane, physical separation is not possible by using existing techniques, and there has been a problem in that it is difficult to identify proteins that are located in each space.

Meanwhile, proximity labeling was developed to reveal proteins or interacting proteins that are present in a specific space, and thereafter, much progress has been made over the past 10 years. This technique proceeds by using an enzyme called BioID, APEX or APEX2 to label proteins within 20 nm of living cells with biotin derivatives, and then lysing the cells and using magnetic beads in which streptavidin is attached to separate only the labeled proteins and perform mass spectrometry, and this innovative method was able to overcome the limitations of existing analysis methods due to contamination during sample production.

In 2014, the Ting group created cells that expressed APEX in the intermembrane space and cells that expressed APEX in the cytoplasm, and cultured the same in a medium containing amino acids labeled with heavy isotopes to reveal 127 mitochondrial intermembrane space proteins (Molecular Cell 55, 1-10, Jul. 17, 2014), but not only was it unable to identify the crista lumen-specific proteins, but also with this method, only amino acids such as lysine (Lys) and arginine (Arg) can be labeled with heavy carbon, and due to the principle of this method, there was a problem in that the protein can be labeled with heavy carbon only by culturing the same in a medium containing heavy carbon-labeled lysine and arginine for a long period of time, and there was also a problem in that the efficiency of the method was very low in live mice. In addition, according to the method, although expensive heavy-carbon labeled lysine and arginine are used to produce many unlabeled heavy-carbon labeled proteins by APEX, there is a problem that causes a lot of economic loss, because these proteins are not used and are discarded.

In order to understand the biochemical phenomena that occur in the peripheral space, which is the intermembrane space of mitochondria, and the cristae lumen, the inventors of the present invention were looking for a way to distinguish between proteins located in the two spaces, and synthesized novel phenolic compounds (heavy biotin-phenol and heavy desthiobiotin-phenol, respectively) in which some of the carbon and nitrogen in biotin-phenol (BP1) and desthiobiotin-phenol (DBP), which are reaction probes for APEX family enzymes that are proximity labeling enzymes, are substituted with non-radioactive isotopes, and the present invention was completed by confirming that when a combination of biotin-phenol/heavy biotin-phenol or desthiobiotin-phenol/heavy desthiobiotin-phenol is used, the quantitative comparative analysis of protein expression in cells in different environments is possible, the analysis of proteins present in spaces that are not separated by membranes, which could not be analyzed previously, is possible, and protein ratios in different adjacent spaces can be quantitatively compared.

### DISCLOSURE

### Technical Problem

An object of the present invention is to provide novel phenolic compounds substituted with non-radioactive isotopes and the uses thereof as probes in proximity molecular labeling.

### Technical Solution

In order to achieve the above object, the present invention provides a phenolic compound represented by Chemical Formula 1 or Chemical Formula 2 below:

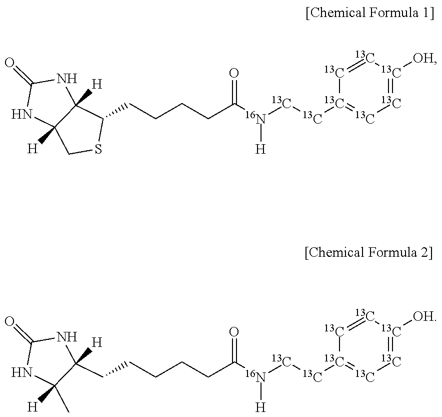

In the present invention, the phenolic compound may be a probe for proximity molecular labeling.

In addition, the present invention provides a method for preparing the compound, including the following steps:
(a) adding pyridoxal-5-phosphate to ¹³C₉ tyrosine or ¹³C₉ ¹⁵N tyrosine to react with tyrosine decarboxylase to respectively produce ¹³C₈ tyramine or ¹³C₈ ¹⁵N tyramine; and
(b) respectively reacting the produced ¹³C₈ tyramine or ¹³C₈ ¹⁵N tyramine with d-Biotinyl-NHS ester or d-Desthiobiotinyl-NHS ester to respectively produce a compound of Chemical Formula 1 or Chemical Formula 2.

In addition, the present invention provides a composition for labeling a protein or peptide, including the phenolic compound represented by Chemical Formula 1 or Chemical Formula 2.

In the present invention, the composition for labeling a protein or peptide may include
(i) biotin phenol and heavy biotin phenol represented by Chemical Formula 1; or
(ii) desthiobiotin phenol and heavy desthiobiotin phenol represented by Chemical Formula 2.

In addition, the present invention provides a method for measuring a protein ratio in adjacent spaces within cells, including the following steps:
(a) expressing (i) a first fusion protein in which an APEX family enzyme is fused with a membrane protein located in a first membrane that separates a first space and a second space within a cell, or (ii) a first fusion protein in which an APEX family enzyme is fused with a protein located in the intermembrane space between a first membrane that separates a first space and a second space and a second membrane that separates a second space and a third space within a cell;
(b) expressing a second fusion protein in which a membrane protein of the second membrane and an APEX family enzyme are fused to the second membrane that separates a second space and a third space within a cell, in a cell that is homologous to the cell expressing the first fusion protein;
(c) treating the cell expressing the first fusion protein with biotin phenol or desthiobiotin phenol, and treating the cell expressing the second fusion protein with heavy biotin phenol represented by Chemical Formula 1 or heavy desthiobiotin phenol represented by Chemical Formula 2;
(d) lysing the cell expressing the first fusion protein and the cell expressing the second fusion protein to separate proteins from each cell; and
(e) mixing the proteins separated from the two cells and measuring the ratio of the biotin- or desthiobiotin-labeled protein and the heavy biotin- or heavy desthiobiotin-labeled protein.

In addition, the present invention provides a method for determining the intracellular location of a protein, including the following steps:
(a) expressing (i) a first fusion protein in which an APEX family enzyme is fused with a membrane protein located in a first membrane that separates a first space and a second space within a cell, or (ii) a first fusion protein in which an APEX family enzyme is fused with a protein located in the intermembrane space between a first membrane that separates a first space and a second space and a second membrane that separates a second space and a third space within a cell;
(b) expressing a second fusion protein in which a membrane protein of the second membrane and an APEX family enzyme are fused to the second membrane that separates a second space and a third space within a cell, in a cell that is homologous to the cell expressing the first fusion protein;
(c) treating the cell expressing the first fusion protein with biotin phenol or desthiobiotin phenol, and treating the cell expressing the second fusion protein with heavy biotin phenol represented by Chemical Formula 1 or heavy desthiobiotin phenol represented by Chemical Formula 2;
(d) lysing the cell expressing the first fusion protein and the cell expressing the second fusion protein to separate proteins from each cell;
(e) mixing the proteins separated from the two cells and measuring the ratio of the biotin- or desthiobiotin-labeled protein and the heavy biotin- or heavy desthiobiotin-labeled protein; and
(f) determining whether the protein is located in the first space, second space or third space within the cell from the ratio of the labeled proteins.

In the present invention, in step (e), the proteins that are separated from the two cells may be mixed at a weight ratio of 1:1.

In the present invention, the first membrane may be an outer mitochondrial membrane (OMM), and the second membrane may be an inner mitochondrial membrane (IMM).

In the present invention, when the first fusion protein is a fusion protein in which a membrane protein located in the first membrane that separates the first space and the second space within the cell is fused with an APEX family enzyme, the protein labeled only by the second fusion protein may be identified as a crista lumen protein.

In the present invention, the first fusion protein of the method may be present in the outer space of the crista lumen (Outer ICS), and the second fusion protein may be present in the intracristal space (ICS).

In the present invention, when the ratio of labeling by the second fusion protein to the ratio of labeling by the first fusion protein is 1.5 or more and the P-value is 0.05 or less, the protein may be identified as a crista lumen protein.

In addition, the present invention provides a method for measuring an intercellular protein ratio, including the following steps:
(a) expressing a fusion protein in which an intracellular protein or membrane protein of a first cell is fused with an APEX family enzyme;
(b) expressing a fusion protein in which an intracellular protein or membrane protein of a second cell is fused with an APEX family enzyme;
(c) treating the first cell with biotin phenol or desthiobiotin phenol, and treating the second cell with heavy biotin phenol represented by Chemical Formula 1 or heavy desthiobiotin phenol represented by Chemical Formula 2;
(d) lysing the first cell and the second cell to separate proteins from each cell; and
(e) mixing the proteins separated from the two cells and measuring the intercellular protein ratio by measuring the ratio of the biotin- or desthiobiotin-labeled protein and the heavy biotin- or heavy desthiobiotin-labeled protein.

In the present invention, the intracellular protein or membrane protein of the first cell and the intracellular protein or membrane protein of the second cell may be homologous proteins.

In the present invention, the intracellular protein may be an intracellular membrane protein.

In the present invention, (i) the first cell may be a normal cell and the second cell may be a diseased cell;
(ii) the first cell may be a diseased cell, and the second cell may be a diseased cell that is treated with a drug; or
(iii) the first cell may be a cell derived from one tissue in vivo, and the second cell may be a cell derived from a different tissue in vivo.

### Advantageous Effects

According to the present invention, proteins that are present in spaces that are not separated by membranes (e.g., mitochondrial cristae lumen), which previously could not be analyzed, can be identified, and the quantitative comparative analysis of protein expression in cells in different environments is possible, and it has the advantage of being able to quantitatively compare and analyze protein ratios in different adjacent spaces, and particularly, it has the advantage of being able to label proteins economically compared to the conventional technique of labeling proteins using heavy-carbon labeled amino acids.

### DESCRIPTION OF DRAWINGS

FIG. 1 shows the structure of a probe used in the present invention (iSpot-ID technique).

FIG. 2 shows the synthesis process of probes (heavy biotin-phenol, heavy desthiobiotin-phenol) used in the present invention.

FIG. 3 is a mimetic diagram showing the process of identifying crista lumen proteins using the present invention. Peptides labeled by APEX that are expressed in the outer and inner mitochondrial membranes can be separated using streptavidin beads, and then, the amount of peptides labeled with different probes can be quantitatively analyzed through mass spectrometry.

FIG. 4 shows the results of identifying crista lumen proteins using the present invention.

FIG. 5 shows the results of verifying by transmission electron microscopy after staining with 3-3′ diaminobenzidine to determine whether TMEM177 and AGK, which were identified as being located in ICS and OCS, are actually located in ICS and OCS through the present invention, respectively.

FIG. 6 shows the results of additional iSPOT-ID analysis using TMEM177-APEX2 and AGK-APEX2 that are identified through the present invention.

FIG. 7a shows the results of verifying by Western blotting whether mitochondria targeting sequence (MTS)-dsRed was actually located in the cristae lumen, in order to verify that mitochondrial matrix proteins were found in the cristae lumen through the additionally performed iSPOT-ID analysis.

FIG. 7b is a mimetic diagram for explaining the principle by which MTS-dsRed, which is a mitochondrial matrix protein, moves into the crista lumen upon tetramer formation.

### MODES OF THE INVENTION

Unless otherwise defined, all technical and scientific terms used in the present specification have the same meaning as commonly understood by a person skilled in the art to which the present invention pertains. In general, the nomenclature used herein and the experimental methods described below are well known and commonly used in the art.

Proximity labeling technology is a technique that uses APEX enzymes that are expressed in a specific space in living cells to label surrounding proteins with a probe and performs mass spectrometry on the labeled proteins to identify proteins located in a specific space. Although the development of proximity labeling technology has made a great leap forward in proteomics, the conventional proximity labeling technique has been used to label proteins in a specific space within a cell or to label proteins in a space that is separated by a membrane, and there have been limitations in identifying proteins in spaces that are not separated by a membrane, such as the cristae lumen and peripheral space of mitochondria, comparing differences in the expression levels of proteins expressed in adjacent spaces within a cell or comparing the differences in expression levels of proteins expressed in cells in different environments.

In the present invention, in order to overcome these limitations, some elements of phenolic compounds that act as probes for APEX family enzymes used in proximity labeling technology are substituted with non-radioactive isotopes to newly synthesize phenolic compounds that have a mass difference of about 9 Da from existing phenolic compounds, and by using the same, it was possible to reveal proteins that are present in the cristae lumen of mitochondria.

Accordingly, in one aspect, the present invention relates to a phenol compound represented by Chemical formula 1 or Chemical Formula 2 below:

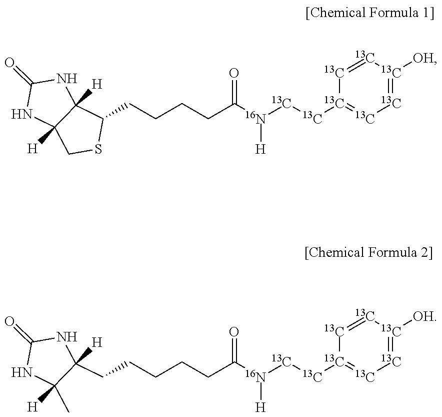

In the present invention, the phenolic compound includes stereoisomers, tautomers or salts thereof.

In the present invention, the phenol compound may be a probe for proximity molecular labeling.

In particular, the phenol compound may be oxidized in the presence of hydrogen peroxide by an APEX family enzyme (APEX or APEX2), which is a proximal molecular labeling enzyme, and label proximal proteins.

Accordingly, in another aspect, the present invention relates to a probe for proximity molecular labeling represented by Chemical Formula 1 or Chemical Formula 2 above.

Meanwhile, in still another aspect, the present invention relates to a method for preparing the compound, including the following steps:
(a) adding pyridoxal-5-phosphate to ¹³C₉ tyrosine or ¹³C₉ ¹⁵N tyrosine to react with tyrosine decarboxylase to respectively produce ¹³C₈ tyramine or ¹³C₈ ¹⁵N tyramine; and
(b) respectively reacting the produced ¹³C₈ tyramine or ¹³C₈ ¹⁵N tyramine with d-Biotinyl-NHS ester or d-Desthiobiotinyl-NHS ester to respectively produce a compound of Chemical Formula 1 or Chemical Formula 2.

Specifically, the present invention may be a method for preparing the compound of Chemical Formula 1, including the following steps:
(a) adding pyridoxal-5-phosphate to ¹³C₉ tyrosine and reacting with tyrosine decarboxylase to produce ¹³C₈ tyramine; and
(b) reacting the produced ¹³C₈ tyramine with d-Biotinyl-NHS ester to produce the compound of Chemical Formula 1.

In addition, the present invention may be a method for preparing the compound of Chemical Formula 2, including the following steps:
(a) adding pyridoxal-5-phosphate to ¹³C₉¹⁵N tyrosine and reacting with tyrosine decarboxylase to produce ¹³C₈¹⁵N tyramine; and
(b) reacting the produced ¹³C₈¹⁵N tyramine with d-Desthiobiotinyl-NHS-ester to produce the compound of Chemical Formula 2.

In the present invention, step (a) may be performed in a buffer of pH 5 to 6, preferably, pH 5.3 to 5.7, at a temperature of about 35 to 39° C., preferably, about 36 to 38° C., for about 4 to 8 hours, preferably, for about 5 to 7 hours. and specifically, step (a) may be characterized by reacting in an acetate buffer of about pH 5.5 at a temperature of about 37° C. for about 6 hours, but the present invention is not limited thereto.

In the present invention, in step (b), triethylamine may be added at a molar ratio of about 1:1 with d-Biotinyl-NHS ester or d-Desthiobiotinyl-NHS-ester, and reacted at room temperature in a solvent of about 3:1 of MeOH:H₂O for about 12 to 20 hours, preferably, about 14 to 18 hours, and most preferably, about 16 hours, but the present invention is not limited thereto.

Ascorbic acid peroxidase (APEX) family enzymes (e.g., APEX, APEX2) oxidize the compound of Chemical Formula 1 or Chemical Formula 2 according to the present invention to phenoxyl radicals in the presence of hydrogen peroxide, and they have the feature of being able to biotin-label proteins or peptides within 20 nm from the enzymes.

Accordingly, in still another aspect, the present invention relates to a composition for labeling a protein or peptide, including the compound of Chemical Formula 1 or Chemical Formula 2.

In the present invention, the composition may be characterized in that the compound is dissolved in a solvent, preferably in cell culture medium or PBS, and treated with cells expressing APEX family enzymes to label proteins or peptides, but the present invention is not limited thereto.

Meanwhile, the compound of Chemical Formula 1 or Chemical Formula 2 according to the present invention may be a component of a kit for labeling a protein or peptide, and therefore, in still another aspect, the present invention may provide a kit for labeling a protein or peptide, including the compound of Chemical Formula 1 or Chemical Formula 2.

In the present invention, the kit is characterized by further including at least selected from the group consisting of hydrogen peroxide, streptavidin beads and kit usage instructions.

When the compound of Chemical Formula 1 or Chemical Formula 2 according to the present invention is used together with biotin-phenol or desthiobiotin-phenol, respectively, the quantitative comparative analysis of protein expression in cells in different environments is possible, and it is possible to analyze proteins present in spaces that are not separated by membranes, which previously could not be analyzed, and the protein ratios in different adjacent spaces can be quantitatively compared.

Therefore, in another aspect, the present invention may be a composition for labeling a protein or peptide, including
(i) biotin phenol and heavy biotin phenol represented by Chemical Formula 1; or
(ii) desthiobiotin phenol and heavy desthiobiotin phenol represented by Chemical Formula 2.

In still another aspect, the present invention may be a composition for quantitative comparative analysis of proteins or peptides, including (i) biotin phenol and heavy biotin phenol represented by Chemical Formula 1; or
(ii) desthiobiotin phenol and heavy desthiobiotin phenol represented by Chemical Formula 2.

In the present invention, the expression level of proteins or peptides in cells in different environments may be compared, from the ratio of proteins or peptides labeled with biotin phenol contained in the composition and proteins or peptides labeled with heavy biotin phenol represented by Chemical Formula 1, and from these results, it is possible to quantitatively confirm the degree of increase or decrease in the expression of a specific protein according to a specific environment (e.g., cancer cells or types of tissues or organs), and since the expression amounts of proteins or peptides that exist in two different spaces that are not separated by a membrane can be compared, these results may quantitatively confirm the degree of increase or decrease in the expression of a specific protein in two different spaces that are not separated by a membrane, or provide information that can determine the exact location information of a specific protein.

Therefore, according to the present invention,
(i) biotin phenol and heavy biotin phenol represented by Chemical Formula 1; or
(ii) desthiobiotin phenol and heavy desthiobiotin phenol represented by Chemical Formula 2 may be a component of a kit for labeling proteins or peptides.

Further, in still another aspect, the present invention may provide (i) biotin phenol and heavy biotin phenol represented by Chemical Formula 1; or
(ii) desthiobiotin phenol and heavy desthiobiotin phenol represented by Chemical Formula 2 in the form of a kit for labeling a protein or peptide or a kit for quantitative comparative analysis of a protein or peptide.

In the present invention, the kit is characterized by further including at least one selected from the group consisting of hydrogen peroxide, streptavidin beads and kit usage instructions.

In one aspect, the present invention may provide a method for measuring a protein ratio in adjacent spaces within cells, including the following steps:
(a) expressing (i) a first fusion protein in which an APEX family enzyme is fused with a membrane protein located in a first membrane that separates a first space and a second space within a cell, or (ii) a first fusion protein in which an APEX family enzyme is fused with a protein located in the intermembrane space between a first membrane that separates a first space and a second space and a second membrane that separates a second space and a third space within a cell;
(b) expressing a second fusion protein in which a membrane protein of the second membrane and an APEX family enzyme are fused to the second membrane that separates a second space and a third space within a cell, in a cell that is homologous to the cell expressing the first fusion protein;
(c) treating the cell expressing the first fusion protein with biotin phenol or desthiobiotin phenol, and treating the cell expressing the second fusion protein with heavy biotin phenol represented by Chemical Formula 1 or heavy desthiobiotin phenol represented by Chemical Formula 2;
(d) lysing the cell expressing the first fusion protein and the cell expressing the second fusion protein to separate proteins from each cell; and
(e) mixing the proteins separated from the two cells and measuring the ratio of the biotin- or desthiobiotin-labeled protein and the heavy biotin- or heavy desthiobiotin-labeled protein.

In the present invention, steps (a) and (b) may be performed in reverse order or simultaneously.

In the present invention, in step (c), it is preferable to treat the cell expressing the second fusion protein with heavy biotin phenol or heavy desthiobiotin phenol, respectively, to correspond to biotin phenol or desthiobiotin phenol treated with the cell expressing the first fusion protein.

In the present invention, step (c) may be characterized in that the cell expressing the first fusion protein is treated with biotin phenol or desthiobiotin phenol, and the cell expressing the second fusion protein is treated with heavy biotin phenol represented by Chemical Formula 1 or heavy desthiobiotin phenol represented by Chemical Formula 2, and then is additional treated with hydrogen peroxide In this case, the hydrogen peroxide may be treated at about 0.1 to 5 mM, preferably, about 0.5 to 2 mM, and most preferably, about 1 mM, and it is preferable that the hydrogen peroxide is treated for about 30 seconds to 10 minutes, for example, about 30 seconds to 2 minutes, but the present invention is not limited thereto.

In the present invention, after step (c), it is preferable to terminate the reaction by treating with 10 mM sodium azide, 10 mM sodium ascorbate and 5 mM trolox.

In the present invention, step (d) may be characterized by lysing the cells using a sonicator, but in another aspect, it may be lysed by using a cell lysis buffer (e.g., RIPA buffer) to which a protease inhibitor cocktail is added.

In the present invention, in step (e), it is preferable to mix proteins at a weight ratio of about 1:1.

In the present invention, step (e) may be characterized by further including the step of isolating biotin- or desthiobintin-labeled protein and heavy biotin- or heavy desthiobiotin-labeled protein, before measuring the ratio of biotin- or desthiobiotin-labeled protein and heavy biotin- or heavy desthiobiotin-labeled protein, and the isolate may be streptavidin, avidin, an analog of streptavidin that reversibly binds to biotin or an analog of avidin that reversibly binds to biotin, but the present invention is not limited thereto.

In the present invention, the method may be characterized by further including the step of digesting the biotin- or desthiobiotin-labeled protein and the heavy biotin- or heavy desthiobiotin-labeled protein with a protease before or after isolation, but the present invention is not limited thereto.

In the present invention, the protease may be selected from the group consisting of trypsin, arginine C (Arg-C), aspartic acid N (Asp-N), glutamic acid C (Glu-C), lysine C (Lys-C), chymotrypsin, Proteinase K and Pronase, but the present invention is not limited thereto.

In the present invention, the biotin- or desthiobiotin-labeled protein and the heavy biotin- or heavy desthiobiotin-labeled protein may be identified by mass spectrometry, but the present invention is not limited thereto.

In the present invention, the mass spectrometer for mass analysis may be selected from the group consisting of LTQ-FT, Orbitrap, Triple-Tof, Q-Tof, Tof-Tof and Q Exactive, but the present invention is not limited thereto.

When the above method is used, the present invention has the advantage of being able to quantitatively compare and analyze the expression level of a specific protein in adjacent spaces within the cell.

In the present invention, the adjacent space within the cell may or may not be separated by a membrane.

In the present invention, in step (c), a person skilled in the art in the present technical field will easily understand that it is possible to perform the experiment by treating the cell expressing the first fusion protein with heavy biotin phenol represented by Chemical Formula 2 or heavy desthiobiotin phenol represented by Chemical Formula 2, and treating the cell expressing the second fusion protein with biotin phenol or desthiobiotin phenol.

In another aspect, the present invention may provide a method for determining the intracellular location of a protein, including the following steps:
(a) expressing (i) a first fusion protein in which an APEX family enzyme is fused with a membrane protein located in a first membrane that separates a first space and a second space within a cell, or (ii) a first fusion protein in which an APEX family enzyme is fused with a protein located in the intermembrane space between a first membrane that separates a first space and a second space and a second membrane that separates a second space and a third space within a cell;
(b) expressing a second fusion protein in which a membrane protein of the second membrane and an APEX family enzyme are fused to the second membrane that separates a second space and a third space within a cell, in a cell that is homologous to the cell expressing the first fusion protein;
(c) treating the cell expressing the first fusion protein with biotin phenol or desthiobiotin phenol, and treating the cell expressing the second fusion protein with heavy biotin phenol represented by Chemical Formula 1 or heavy desthiobiotin phenol represented by Chemical Formula 2;
(d) lysing the cell expressing the first fusion protein and the cell expressing the second fusion protein to separate proteins from each cell;
(e) mixing the proteins separated from the two cells and measuring the ratio of the biotin- or desthiobiotin-labeled protein and the heavy biotin- or heavy desthiobiotin-labeled protein; and
(f) determining whether the protein is located in the first space, second space or third space within the cell from the ratio of the labeled proteins.

In the method for determining the intracellular location of a protein according to the present invention, steps (a) to (e) may be carried out in the same process as steps (a) to (e) in the method for measuring a protein ratio in the adjacent space within the cell, or may be carried out in a modified process to the extent that steps (a) to (e) can be easily modified and implemented by a person skilled in the art in the present technical field.

In the present invention, step (e) may be characterized in that the proteins separated from the two cells are mixed at a weight ratio of about 1:1.

In the present invention, the first membrane may be an outer mitochondrial membrane (OMM), and the second membrane may be an inner mitochondrial membrane (IMM), but the present invention is not limited thereto.

In this case, when the first fusion protein is a fusion protein in which a membrane protein located in the first membrane that separates the first space and the second space within the cell is fused with an APEX family enzyme, the method may be characterized by identifying a protein labeled only by the second fusion protein as a crista lumen protein, but the present invention is not limited thereto.

In the present invention, the first fusion protein may be present in the outer space of the crista lumen (Outer ICS), and the second fusion protein may be present in the intracristal space (ICS).

In the present invention, a protein having a ratio of being labeled by the second fusion protein of 1.5 or more compared to the ratio of being labeled by the first fusion protein may be characterized as a crista lumen protein, but the present invention is not limited thereto.

In this case, a protein with a P-value of 0.05 or less may be characterized as a crista lumen protein, but the present invention is not limited thereto.

In one aspect, when the first fusion protein is a fusion protein in which a membrane protein located in the first membrane that separates the first space and the second space within the cell is fused with an APEX family enzyme, the method may be characterized by identifying a protein labeled only by the second fusion protein as a crista lumen protein, when comparing a biotin-phenol labeled protein/heavy biotin-phenol labeled protein or a desthiobiotin-phenol labeled protein/heavy desthiobiotin-phenol labeled protein.

In another aspect, when the first fusion protein is a fusion protein in which a protein located between the first space and the second space in the cell is fused with an APEX family enzyme, the method may be characterized by identifying a protein with a biotin-phenol labeled protein (HBP/LBP) ratio of 1.5 and a P value of 0.05 or less as a crista lumen protein, compared to a protein labeled with heavy desthiobiotin-phenol, a protein labeled with desthiobiotin-phenol (HDBP/LDBP) or a protein labeled with heavy biotin-phenol Specifically, the present invention may provide a method for identifying a crista luminal protein, including the following steps:
(a) expressing a first fusion protein in which an outer mitochondrial membrane protein and an APEX family enzyme are fused to the outer mitochondrial membrane (OMM), which separates the intracellular cytosol and the mitochondrial intermembrane space (IMS);
(b) expressing a second fusion protein in which an inner mitochondrial membrane protein and an APEX family enzyme are fused to the inner mitochondrial membrane (IMM), which separates the intracellular mitochondrial intermembrane space (IMS) and the mitochondrial matrix;
(c) treating the cell expressing the first fusion protein with desthiobiotin phenol and treating the cell expressing the second fusion protein with desthiobiotin phenol represented by Chemical Formula 2;
(d) lysing the cell expressing the first fusion protein and the cell expressing the second fusion protein to separate proteins from each cell;
(e) mixing the proteins separated from the two cells and measuring the ratio of the desthiobiotin-labeled protein and the desthiobiotin-labeled protein; and (f) identifying the protein as a crista lumen protein, when the protein is labeled only by the second fusion protein among the labeled proteins.

In another aspect, the present invention may provide a method for identifying a crista luminal protein, including the following steps:
(a) expressing a first fusion protein in which an outer mitochondrial membrane protein and an APEX family enzyme are fused to the outer mitochondrial membrane (OMM), which separates the intracellular cytosol and the mitochondrial intermembrane space (IMS);
(b) expressing a second fusion protein in which an inner mitochondrial membrane protein and an APEX family enzyme are fused to the inner mitochondrial membrane (IMM), which separates the intracellular mitochondrial intermembrane space (IMS) and the mitochondrial matrix;
(c) treating the cell expressing the first fusion protein with biotin phenol and treating the cell expressing the second fusion protein with heavy biotin phenol represented by Chemical Formula 2;
(d) lysing the cell expressing the first fusion protein and the cell expressing the second fusion protein to separate proteins from each cell;
(e) mixing the proteins separated from the two cells and measuring the ratio of biotin-labeled proteins and heavy biotin-labeled proteins; and
(f) identifying the protein as a crista lumen protein, when the protein is labeled only by the second fusion protein among the labeled proteins.

In the present invention, the outer mitochondrial membrane protein in step (a) may be TDRKH, and the inner mitochondrial membrane protein in step (b) may be SCO1, but the present invention not limited thereto.

In the present invention, in the first fusion protein, the APEX family enzyme may be fused to be expressed in the mitochondrial intermembrane space (IMS), and in the second fusion protein, the APEX family enzyme may be fused to be expressed in the cytoplasm, but the present invention is not limited thereto.

In another aspect, the present invention may provide a method for identifying a crista luminal protein, including the following steps:
(a) expressing a first fusion protein in which a protein located in the intracellular mitochondrial intermembrane space (IMS) is fused with an APEX family enzyme;
(b) expressing a second fusion protein in which an inner mitochondrial membrane protein and an APEX family enzyme are fused to the inner mitochondrial membrane (IMM), which separates the intracellular mitochondrial intermembrane space (IMS) and the mitochondrial matrix;
(c) treating the cell expressing the first fusion protein with desthiobiotin phenol and treating the cell expressing the second fusion protein with desthiobiotin phenol represented by Chemical Formula 2;
(d) lysing the cell expressing the first fusion protein and the cell expressing the second fusion protein to separate proteins from each cell;
(e) mixing the proteins separated from the two cells and measuring the ratio of desthiobiotin-labeled proteins and desthiobiotin-labeled proteins; and
(f) identifying a protein whose ratio of being labeled by the first fusion protein to the ratio of being labeled by the second fusion protein is about 1.5 or more as a crista lumen protein.

In another aspect, the present invention may provide a method for identifying a crista luminal protein, including the following steps:
(a) expressing a first fusion protein in which a protein located in the intracellular mitochondrial intermembrane space (IMS) is fused with an APEX family enzyme;
(b) expressing a second fusion protein in which an inner mitochondrial membrane protein and an APEX family enzyme are fused to the inner mitochondrial membrane (IMM), which separates the intracellular mitochondrial intermembrane space (IMS) and the mitochondrial matrix;
(c) treating the cell expressing the first fusion protein with biotin phenol and treating the cell expressing the second fusion protein with heavy biotin phenol represented by Chemical Formula 2;
(d) lysing the cell expressing the first fusion protein and the cell expressing the second fusion protein to separate proteins from each cell;
(e) mixing the proteins separated from the two cells and measuring the ratio of biotin-labeled proteins and heavy biotin-labeled proteins; and
(f) identifying a protein whose ratio of being labeled by the first fusion protein to the ratio of being labeled by the second fusion protein is about 1.5 or more as a crista lumen protein.

In the present invention, the protein located in the intracellular mitochondrial intermembrane space (IMS) in step (a) may be AGK, and the internal mitochondrial membrane protein in step (b) may be TMEM177, but the present invention is not limited thereto.

In the present invention, the APEX family enzymes in the first and second fusion proteins may be characterized in that they are fused to be expressed in the mitochondrial intermembrane space (IMS), but the present invention is not limited thereto.

In another aspect, the present invention may provide a method for measuring an intercellular protein ratio, including the following steps:
(a) expressing a fusion protein in which an intracellular protein or membrane protein of a first cell is fused with an APEX family enzyme;
(b) expressing a fusion protein in which an intracellular protein or membrane protein of a second cell is fused with an APEX family enzyme;
(c) treating the first cell with biotin phenol or desthiobiotin phenol, and treating the second cell with heavy biotin phenol represented by Chemical Formula 1 or heavy desthiobiotin phenol represented by Chemical Formula 2;
(d) lysing the first cell and the second cell to separate proteins from each cell; and
(e) mixing the proteins separated from the two cells and measuring the intercellular protein ratio by measuring the ratio of the biotin- or desthiobiotin-labeled protein and the heavy biotin- or heavy desthiobiotin-labeled protein.

In the present invention, the intracellular protein or membrane protein of the first cell and the intracellular protein or membrane protein of the second cell may be characterized as homologous proteins.

In the present invention, the intracellular protein may be characterized as an intracellular membrane protein, but the present invention is not limited thereto.

In the present invention,
(i) the first cell may be a normal cell and the second cell may be a diseased cell;
(ii) the first cell may be a diseased cell and the second cell may be a diseased cell treated with a drug; or
(iii) the first cell may be a cell derived from one tissue in the living body and the second cell may be a cell derived from a different tissue in the living body, but the present invention is not limited thereto.

In the present invention, the diseased cells may be cancer cells.

In the present invention, cancer cells may be selected from squamous cell cancer, small cell lung cancer, non-small cell lung cancer, lung adenocarcinoma, lung cancer, peritoneal cancer, hepatocellular cancer, gastrointestinal cancer, pancreatic cancer, glioblastoma, cervical cancer, ovarian cancer, liver cancer, bladder cancer, liver cancer, breast cancer, colon cancer, colorectal cancer, endometrial or uterine cancer, salivary gland cancer, kidney cancer, liver cancer, prostate cancer, vulvar cancer, thyroid cancer, liver cancer and various head and neck cancers, but the present invention is not limited thereto.

In the present invention, the tissue may be selected from cartilage, bone, blood vessels, brain, liver, heart, ligament, muscle, spinal cord, blood, bone marrow, lung, teeth, nerve, cornea, retina, esophagus, spine, kidney, pancreas and urethra, but the present invention is not limited thereto.

The present invention has the advantage of being able to quantitatively compare and analyze the expression level of a specific protein between cells in different environments.

By using the compound according to the present invention, the present invention was able to identify 56 proteins that are specifically present in the intracristal space (ICS) that have been previously unknown.

In addition, TMEM177, which is one of the proteins identified as crista lumen proteins through the method of the present invention, and AGK protein, which was confirmed to exist in the peripheral space, were used to compare microscopic environments in the crista lumen and peripheral space or outer-ICS).

In all of the above methods, a person skilled in the art would be able to treat the biotin phenol and heavy biotin phenol in a vice versa manner and then proceed with the experiment by easily modifying the biotin phenol appropriately. Similarly, a person skilled in the art may treat the desthiobiotin phenol and desthiobiotin phenol in a vice versa manner and then proceed with the experiment by easily modifying the same appropriately.

As used herein, the term “about” is used to mean approximately, roughly, around or in the regions of. When the term “about” is used with a numerical range, the range is modified by extending the boundaries above and below the stated numerical value. Generally, the term “about” is used to modify a stated value up or down (higher or lower) by a variation of 10% to a numerical value above or below.

As used herein, the term “protein” includes peptides.

### EXAMPLE

Hereinafter, the present invention will be described in more detail through examples. These examples are only for illustrating the present invention, and it will be apparent to those skilled in the art that the scope of the present invention is not to be construed as limited by these examples.

### Example 1. Preparation of Heavy-Carbon Substituted Probes

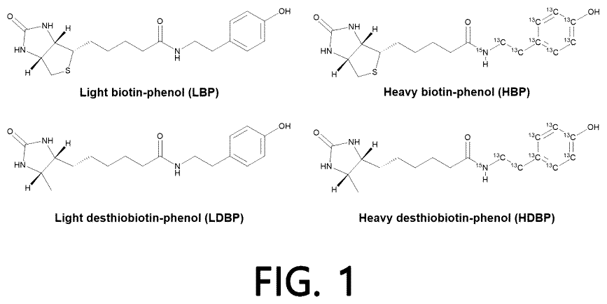

In the present invention, compounds in which biotin-phenol (BP) and desthiobiotin-phenol (DBP), which are the conventional reactive probes of APEX2, which is one of the enzymes in proximity labeling, were respectively substituted with heavy carbon were newly synthesized, and the experiments were conducted by using a total of four probes, that is, iBP (BP, HBP) and iDBP (DBP, HDBP) (FIG. 1). In order to distinguish from HBP and HDBP, BP and DBP, which have been conventionally used, are denoted as LBP and LDBP, respectively, or simply as L throughout the specification, including FIG. 1. LBP and LDBP were preferred by referring to existing research articles (Ntai, I.; Phelan, V. V.; Bachmann, B. O., Phosphonopeptide K-26 biosynthetic intermediates in Astrosporangium hypotensionis. Chemical Communications 2006, (43), 4518-4520).

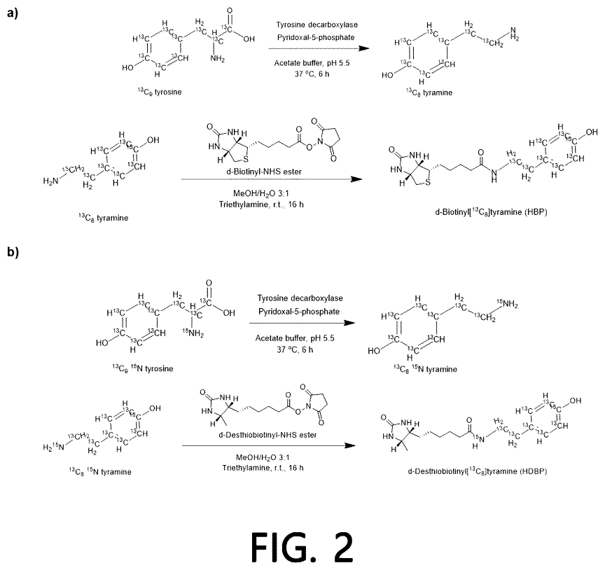

The preparation methods of HBP and HDBP used in the present invention are as follows (FIG. 2).

100 mg of [¹³C₉] tyrosine (Cambridge Isotope Laboratory) was added to 100 mL of water and heated in a microwave oven until it was completely dissolved. After cooling to room temperature again, 2 mg of tyrosine decarboxylase apoenzyme (0.05 unit/mg, Sigma Aldrich) obtained from Streptococcus faecalis and 7 mg of pyridoxal-5-phosphate were dissolved in acetate buffer (4 ml, 0.1M, pH 5.5) and added. After stirring at 37° C. for 6 hours, heat was applied to terminate the reaction. The reaction material was concentrated by using Dowex 50WX cation exchange resin (hydrogen form), and [¹³C₈] tyramine was separated by using 1 to 10 mM HCl.
100 mg of [¹³C₈] tyramine was dissolved in 5 mL of methanol/distilled water (3:1), and 204 mg of d-Biotinyl-NHS ester (sigma Aldrich) and 83 μL of triethylamine (sigma Aldrich) were added. After adding 5 mL of methanol and reacting at room temperature for 16 hours, the reaction material was concentrated in vacuum and extracted from water by using 50 mL of ethyl acetate (Samchun Chemical). The extracted organic layer materials were added with magnesium sulfate (Daejeong Chemical), filtered (ADVANTEC) and then concentrated in vacuum. Again, the reaction materials were separated through column chromatography filled with silica gel 60 (0.04 to 0.063 mm) (Merck), and HBP was obtained under the condition of acetate:methanol=5:1.

In order to synthesize HDBP, 100 mg of [¹³C₉, ¹⁵N] tyrosine was used as a starting material, and reaction and separation were performed under the same conditions as HBP to obtain [¹³C₈, ¹⁵N] tyramine. 190.8 mg of [¹³C₈, ¹⁵N] tyramine was dissolved in 5 mL of methanol/distilled water (3:1), and 450.1 mg of d-Desthiobiotinyl-NHS ester and 209 L of triethylamine were added. 5 mL of methanol was added, and after the reaction was carried out at room temperature for 16 hours, the reaction material was concentrated in vacuum and extracted from water by using 50 mL of ethyl acetate. The extracted organic layer materials were added with magnesium sulfate, filtered and then concentrated in vacuum. Reaction materials were separated through reverse-phase HPLC (C18 column, 10 mL/min, 15 to 35% acetonitrile/water over 40 min, retention time 10 min).

### Example 2. Identification of Crista Lumen Proteins Through iSPOT-ID Analysis

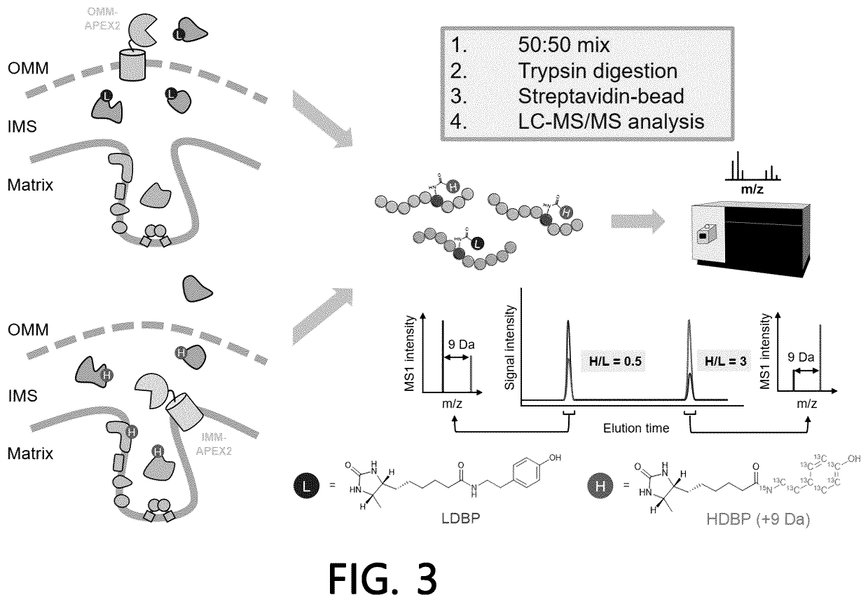

FIG. 3 is a schematic diagram showing the experimental process for iSpot-ID analysis to identify crista lumen proteins.

Specifically, in order to fabricate cells expressing APEX family enzymes on the outer mitochondrial membrane (OMM) (TDRKH(OMM)-APEX) or cells expressing APEX family enzymes on the inner mitochondrial membrane (IMM) (SCO1(IMM)-APEX), respectively, the Flp-In™ T-REx™ 293 cell line (Thermofisher) was cultured in Dulbecco Modified Eagle Medium (DMEM, Falcon) containing 10% fetal bovine serum (FBS, Atlas Biologicals) under the conditions of 5% CO₂ and 37° C. Thereafter, after cloning the TDRKH-V5-APEX2 and SCO1-V5-APEX2 sequences into the pCDNA5 vector (Thermofisher) by using KpnI/NotI restriction enzymes (NEB) and ligase (NEB), it was expressed through transfection using polyethylenimine (PEI) with the pOG44 vector and integrated into the FRT site of the genomic DNA of the Flp-In™ T-REx™ 293 cell line.

SEQ ID NO: 1: TDRKH-V5-APEX2-AP sequence
atgtctactgaacggacttcttggacaagcctgtccaccattcag
aaaatagccctgggccttgggatcccagccagtgcaacagttgcc
tatatcctataccgcaggtatagggaaagcagagaagagcggctg
acatttgttggggaagatgacattgagatagagatgcgggttccc
caggaggctgtgaaactcatcattggccggcaaggagccaatatt
aaacagctgcggaaacagacaggtgctcggattgatgtggacaca
gaggatgtaggcgatgagcgagtgctgcttatcagtggttttcct
gttcaggtgtgcaaggccaaagcagcaatccatcagatcctgaca
gagaataccccagtgtctgagcagctttcagttccccagagatct
gtgggcagaatcatagggagaggcggcgagacaattcgttctatc
tgtaaggcatctggagccaaaattacctgtgacaaagaatcagaa
gggacattactactatcaagacttataaaaatctcaggaacacag
aaggaagtggcagcagccaagcatttgatactggagaaagtttca
gaagatgaagaacttcggaagagaattgctcattctgcagaaacc
agggtcccacgcaaacagccaatcagtgtgagaagagaagacatg
acagagccaggtggagctggagagccagcattatggaaaaacacc
agttctagcatggagccgactgcacccctggtgactcctccaccc
aaaggaggaggcgacatggctgtggtagtgtcaaaggaaggttcc
tgggagaaacctagtgatgacagctttcagaagtctgaagcccag
gccatcccagagatgcccatgtttgaaatccccagtcctgacttc
agttttcatgctgatgagtacctagaagtctacgtttctgcttct
gagcaccctaaccacttctggatccagatcgttggctcccgcagc
ctgcaattggataagcttgtcaatgagatgacccagcactatgag
aatagtgtgcctgaagacttgactgtgcatgtaggagacattgta
gcagcacctttacctacaaatggttcctggtatcgagcccgggtc
ctcggcaccttggagaatgggaacttggacctctattttgttgac
tttggagataatggagattgcccactgaaggacctcagggctctc
aggagtgacttcctaagccttccatttcaagcaatagaatgtagt
ctggcacggattgctccctcaggtgaccagtgggaagaggaagct
ttggatgagtttgatagactcactcattgtgctgactggaagcct
ctggtagccaagatctctagctatgtccagactgggatctcaact
tggccaaagatctacttatatgatactagcaatgggaagaaactt
gatattgggctagaattagtacacaaaggatacgcaattgagctt
cctgaagacatagaagaaaacagagctgtcccagacatgttgaag
gacatggccacagaaacagatgcctctctcagcacgttgctcact
gagaccaaaaagagctctggagagataacacataccctgtcctgc
ctcagcttatcagaagctgcttccatgtctggtgatgataacctt
gaagatgactacttactcaaggatccGCTAGCaGGCAAGCCCATC
CCCAACCCCCTGCTGGGCCTGGACAGCACCGGAAAGTCTTACCCA
ACTGTGAGTGCTGATTACCAGGACGCCGTTGAGAAGGCGAAGAAG
AAGCTCAGAGGCTTCATCGCTGAGAAGAGATGCGCTCCTCTAATG
CTCCGTTTGGCATTCCACTCTGCTGGAACCTTTGACAAGGGCACG
AAGACCGGTGGACCCTTCGGAACCATCAAGCACCCTGCCGAACTG
GCTCACAGCGCTAACAACGGTCTTGACATCGCTGTTAGGCTTTTG
GAGCCACTCAAGGCGGAGTTCCCTATTTTGAGCTACGCCGATTTC
TACCAGTTGGCTGGCGTTGTTGCCGTTGAGGTCACGGGTGGACCT
AAGGTTCCATTCCACCCTGGAAGAGAGGACAAGCCTGAGCCACCA
CCAGAGGGTCGCTTGCCCGATCCCACTAAGGGTTCTGACCATTTG
AGAGATGTGTTTGGCAAAGCTATGGGGCTTACTGACCAAGATATC
GTTGCTCTATCTGGGGGTCACACTATTGGAGCTGCACACAAGGAG
CGTTCTGGATTTGAGGGTCCCTGGACCTCTAATCCTCTTATTTTC
GACAACTCATACTTCACGGAGTTGTTGAGTGGTGAGAAGGAAGGT
CTCCTTCAGCTACCTTCTGACAAGGCTCTTTTGTCTGACCCTGTA
TTCCGCCCTCTCGTTGATAAATATGCAGCGGACGAAGATGCCTTC
TTTGCTGATTACGCTGAGGCTCACCAAAAGCTTTCCGAGCTTGGG
TTTGCTGATGCCggcctgaacgacatcttcgaggcccagaagatc
gagtggcacgag
SEQ ID NO: 2:
SCO1-V5-APEX2 sequence
ATGGCGATGCTGGTCCTAGTACCCGGACGAGTTATGCGGCCTCTG
GGTGGCCAACTTTGGCGCTTCTTGCCTCGCGGACTCGAGTTTTGG
GGCCCAGCCGAGGGGACTGCGAGAGTCTTGCTGAGGCAGTTCTGC
GCGCGGCAAGCGGAGGCGTGGCGTGCCTCGGGGCGCCCTGGCTAT
TGCCTGGGAACCCGGCCCCTCAGCACTGCGAGGCCGCCACCCCCG
TGGTCGCAGAAGGGCCCCGGAGACTCCACGCGCCCCTCGAAGCCC
GGGCCTGTTTCCTGGAAGTCTTTAGCAATCACATTTGCTATTGGA
GGAGCTTTACTGGCTGGAATGAAGCACGTCAAGAAAGAAAAGGCA
GAGAAGTTAGAGAAGGAACGGCAGCGACACATCGGCAAGCCTTTA
CTTGGGGGACCGTTTTCCCTCACAACTCATACTGGGGAGCGTAAA
ACTGACAAGGACTACTTGGGTCAGTGGTTATTGATTTATTTTGGC
TTCACTCATTGCCCTGATGTCTGTCCAGAAGAACTAGAAAAGATG
ATTCAAGTCGTGGATGAAATAGATAGCATTACAACTCTGCCAGAT
CTAACTCCACTTTTCATCAGCATTGACCCAGAGAGGGACACAAAA
GAAGCCATCGCAAATTATGTGAAAGAATTTTCTCCCAAACTGGTT
GGCTTGACTGGCACGAGAGAAGAGGTCGATCAAGTGGCCAGAGCA
TACAGAGTGTATTACAGCCCTGGCCCCAAGGACGAAGATGAAGAC
TACATAGTGGATCACACAATAATAATGTACTTGATTGGACCAGAT
GGTGAGTTTCTAGATTATTTTGGCCAGAACAAGAGGAAGGGAGAA
ATAGCTGCTTCAATTGCCACACACATGAGGCCATACAGAAAAAAG
AGCaaggatccaGGCAAGCCCATCCCCAACCCCCTGCTGGGCCTG
GACAGCACCGGAAAGTCTTACCCAACTGTGAGTGCTGATTACCAG
GACGCCGTTGAGAAGGCGAAGAAGAAGCTCAGAGGCTTCATCGCT
GAGAAGAGATGCGCTCCTCTAATGCTCCGTTTGGCATTCCACTCT
GCTGGAACCTTTGACAAGGGCACGAAGACCGGTGGACCCTTCGGA
ACCATCAAGCACCCTGCCGAACTGGCTCACAGCGCTAACAACGGT
CTTGACATCGCTGTTAGGCTTTTGGAGCCACTCAAGGCGGAGTTC
CCTATTTTGAGCTACGCCGATTTCTACCAGTTGGCTGGCGTTGTT
GCCGTTGAGGTCACGGGTGGACCTAAGGTTCCATTCCACCCTGGA
AGAGAGGACAAGCCTGAGCCACCACCAGAGGGTCGCTTGCCCGAT
CCCACTAAGGGTTCTGACCATTTGAGAGATGTGTTTGGCAAAGCT
ATGGGGCTTACTGACCAAGATATCGTTGCTCTATCTGGGGGTCAC
ACTATTGGAGCTGCACACAAGGAGCGTTCTGGATTTGAGGGTCCC
TGGACCTCTAATCCTCTTATTTTCGACAACTCATACTTCACGGAG
TTGTTGAGTGGTGAGAAGGAAGGTCTCCTTCAGCTACCTTCTGAC
AAGGCTCTTTTGTCTGACCCTGTATTCCGCCCTCTCGTTGATAAA
TATGCAGCGGACGAAGATGCCTTCTTTGCTGATTACGCTGAGGCT
CACCAAAAGCTTTCCGAGCTTGGGTTTGCTGATGCC

Afterwards, only integrated cells were selected by using hygromycin (Thermofisher) to create a stable cell line. Each cell line was cultured in a T75 flask, treated with 5 ng/mL doxycycline (Sigma Aldrich) to express APEX2, and then treated with DMEM containing 250 μM of biotin-phenol (BP) or heavy biotin-phenol (HBP) for 30 minutes, and thereafter, hydrogen peroxide (Sigma Aldrich) was treated at a concentration of 1 mM for 1 minute.

Afterwards, phosphate buffered saline (PBS) in which 10 mM sodium azide (Sigma Aldrich), sodium ascorbate (Sigma Aldrich) and 5 mM trolox (Sigma Aldrich) were dissolved was treated to terminate the reaction.

After removing the cells from the flask using a cell scraper, the cells were precipitated at a speed of 700 rcf for 5 minutes to pellet the cells. For cell lysis, the cells were treated with 750 μL of 2% SDS dissolved in 1× Tris-buffered saline (TBS, Thermofisher) supplemented with protease inhibitor mixture (Thermofisher), respectively. After each cell was completely lysed by using a sonicator (Bioruptor) at a frequency of 20 to 40 kHz for 15 minutes, 4 mL of acetone was added to precipitate the protein. At least 2 hours later, the total protein was dissolved in 50 mM ammonium bicarbonate (ABC, sigma aldrich) buffer containing 8M urea (Sigma Aldrich) and quantified by using a BCA assay kit (Thermofisher).

The proteins separated from the two cells were mixed at a weight ratio of 1:1 to make a total of 6 mg. Afterwards, the proteins were denatured at 37° C. for 1 hour, reduced with 10 mM dithiothreitol (DTT, Sigma Aldrich), and then alkylated with 55 mM iodoacetamide (IAM, Sigma Aldrich). Urea concentration was lowered to 1 mM by adding 50 mM ABC buffer, and 120 g of trypsin (Thermofisher) was treated at 37° C. for 12 hours.

Afterwards, in order to isolate only the labeled peptide, the peptide solution was incubated with 150 μL of streptavidin beads that non-covalently bonded with DBP or HDBP for 1 hour, and then washed twice with 50 mM ABC buffer containing 2M urea. After washing once more with distilled water for mass spectrometry to remove salt, the labeled peptide was treated with 150 μL of an eluent solution consisting of 80% acetonitril (ACN, Sigma aldrich), 19.7% distilled water for mass spectrometry (Sigma aldrich), 0.2% trifluoroacetic acid (TFA, Thermofisher) and 0.1% formic acid (FA, Thermofisher) 3 times at 60° C. for 5 minutes to extract the labeled peptide from the beads.

Meanwhile, in still another aspect, an additional experiment was conducted in the same manner as the above experiment by modifying only the probes into desthiobiotin phenol (DBP) and desthiobiotin phenol (HDBP), respectively.

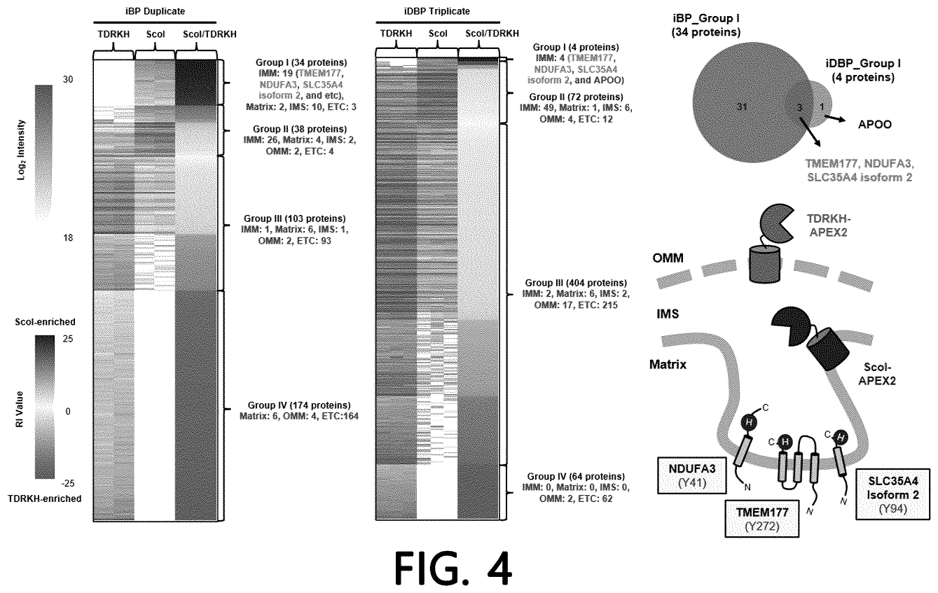

The results are as shown in FIG. 4, and in the results of analysis using biotin-phenol/heavy biotin-phenol (iBP probe), although the proteins labeled by TDRKH(OMM)-APEX and SCO1(IMM)-APEX did not overlap more than half of each other, but in the results of analysis using desthiobiotin-phenol/heavy desthiobiotin-phenol (iDBP probe), it was confirmed that most proteins were labeled with both of desthiobiotin-phenol and desthiobiotin-phenol. This is determined to be due to the lower yield of the iBP probe compared to the iDBP probe when eluting from streptavidin beads, and therefore, it was found that the accuracy could be improved when using the iDBP probe compared to using the iBP probe.

By analyzing these results, the protein that was labeled in heavy desthiobiotin-phenol or heavy biotin-phenol but not in desthiobiotin-phenol or biotin-phenol was determined to be a crista lumen protein. Specifically, it was confirmed that three proteins (TMEM177, NDUFA3 and SLC35A4 isoform2) were labeled only by SCO1(IMM)-APEX2.

### Example 3. Verification of iSPOT-ID Analysis Results

In order to confirm whether TMEM177 and AGK identified in Example 2 are located in the ICS and OCS, respectively, stable cell lines expressing TMEM177-APEX2 and AGK-APEX2, respectively, were produced in the same manner as in Example 2. To this end, SCO1 was removed from pCDNA5 vector including SCO1-V5-APEX2 using KpnI and NheI restriction enzymes and replaced with TMEM177 and AGK sequences and cloned. After APEX2 was expressed by treating the cells with doxycycline for 24 hours, the cells were fixed by treating the same with 4% paraformaldehyde for 15 minutes. Paraformaldehyde was removed by replacing the same with PBS three times, and then, the cells were treated with a PBS solution including 3-3′ diaminobenzidine (Sigma Aldrich) at a concentration of 0.5 mg/mL and 10 mM H₂O₂, stained for 10 minutes and observed under a transmission electron microscope.

SEQ ID NO: 3:
TMEM177 sequence
atggcaggtcccctgtggcggaccgcagcatttgtgcagagacac
aggacaggcctcttggtgggttcctgtgcaggcctgtttggagtt
ccagtctcgtaccacctcttcccggatcccgtggtccaatggctc
taccagtactggcctcagggccagccagctccgctccctccacag
ctgcagagcctcttccaagaggtgctacaggacataggtgttcct
tcaggccattgctacaagcccttcaccaccttcaccttccagcct
gtgagtgcaggcttcccaagactccctgctggggctgtggtgggc
atccctgccagtttcttgggagacctagtgatcaacactaaccat
cccgtggtcatacatgggcatacagtggactggcggagcccagca
ggcgcccggctgagagcttccctgaccttgtcccgtgaagcccag
aagttcgccttggccagggaagtggtgtacctggaaagcagtacc
actgccgtgcacgccctgctggccccagcttgcctggcagggacc
tgggcactgggcgtgggtgccaagtacaccctggggctccatgca
ggccccatgaatttacgggctgccttcagcttggtggcagcagtg
gcaggctttgtggcctacgccttctcccaggattctctcactcat
gccgtggagtcctggctggaccgccgcacggcctccctctctgca
gcctatgcctgtggtggagtggagttctatgagaagcttctgtcg
ggcaacctggccctgcgcagtctcttgggcaaagagggggagaag
ctgtatacacccagcgggaacatcgtccccagacacttgttccga
atcaaacatttaccctacaccacccgccgggactctgtgctgcag
atgtggagggggatgctcaatccgggccgctcc
SEQ ID NO: 4:
AGK sequence
atgacggtgttctttaaaacgcttcgaaatcactggaagaaaact
acagctgggctctgcctgctgacctggggaggccattggctctat
ggaaaacactgtgataacctcctaaggagagcagcctgtcaagaa
gctcaggtgtttggcaatcaactcattcctcccaatgcacaagtg
aagaaggccactgtttttctcaatcctgcagcttgcaaaggaaaa
gccaggactctatttgaaaaaaatgctgccccgattttacattta
tctggcatggatgtgactattgttaagacagattatgagggacaa
gccaagaaactcctggaactgatggaaaacacggatgtgatcatt
gttgcaggaggagatgggacactgcaggaggttgttactggtgtt
cttcgacgaacagatgaggctaccttcagtaagattcccattgga
tttatcccactgggagagaccagtagtttgagtcataccctcttt
gccgaaagtggaaacaaagtccaacatattactgatgccacactt
gccattgtgaaaggagagacagttccacttgatgtcttgcagatc
aagggtgaaaaggaacagcctgtatttgcaatgaccggccttcga
tggggatctttcagagatgctggcgtcaaagttagcaagtactgg
tatcttgggcctctaaaaatcaaagcagcccactttttcagcact
cttaaggagtggcctcagactcatcaagcctctatctcatacacg
ggacctacagagagacctcccaatgaaccagaggagacccctgta
caaaggccttctttgtacaggagaatattacgaaggcttgcgtcc
tactgggcacaaccacaggatgccctttcccaagaggtgagcccg
gaggtctggaaagatgtgcagctgtccaccattgaactgtccatc
acaacacggaataatcagcttgacccgacaagcaaagaagatttt
ctgaatatctgcattgaacctgacaccatcagcaaaggagacttt
ataactataggaagtcgaaaggtgagaaaccccaagctgcacgtg
gagggcacggagtgtctccaagccagccagtgcactttgcttatc
ccggagggagcagggggctcttttagcattgacagtgaggagtat
gaagcgatgcctgtggaggtgaaactgctccccaggaagctgcag
ttcttctgtgatcctaggaagagagaacagatgctcacaagcccc
acccag

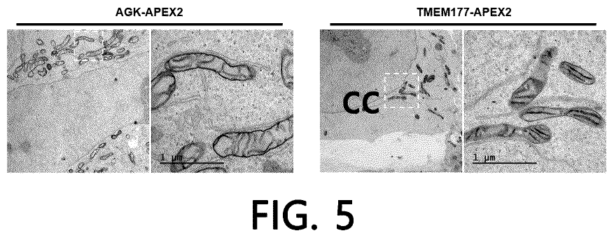

As a result, it was confirmed that TMEM177 and AGK were located in the ICS and OCS, respectively, as shown in FIG. 5.

### Example 4. iSPOT-ID Analysis Using TMEM177

In another aspect to identify the crista lumen proteome, iSPOT-ID analysis was further performed by using the TMEM177-APEX2 and AGK-APEX2 cell lines produced in Example 3. The experimental method was conducted in the same manner as Examples 2 and 3, except for the expressed fusion protein.

Cell lines expressing TMEM177-APEX2 were treated with DBP, and cell lines expressing AGK-APEX2 were treated with HDBP.

In TMEM177-APEX2, TMEM177 is an inner mitochondrial transmembrane protein that passes through the inner mitochondrial membrane and is expressed in the cristae lumen, and APEX2 is fused to be expressed in the mitochondrial intermembrane space (IMS). In AGK-APEX2, AGK exists in the space outside the crista lumen in the IMS area, and AGK-APEX2 is expressed in the IMS, and thus, the ratio of proteins labeled in desthiobiotin-phenol or biotin-phenol to the proteins labeled in heavy desthiobiotin-phenol or heavy biotin-phenol was calculated (i.e., HDBP/LDBP or HBP/LBP), and when Fold change >1.5 (P-Value<0.05), it was identified as a crista luminal protein.

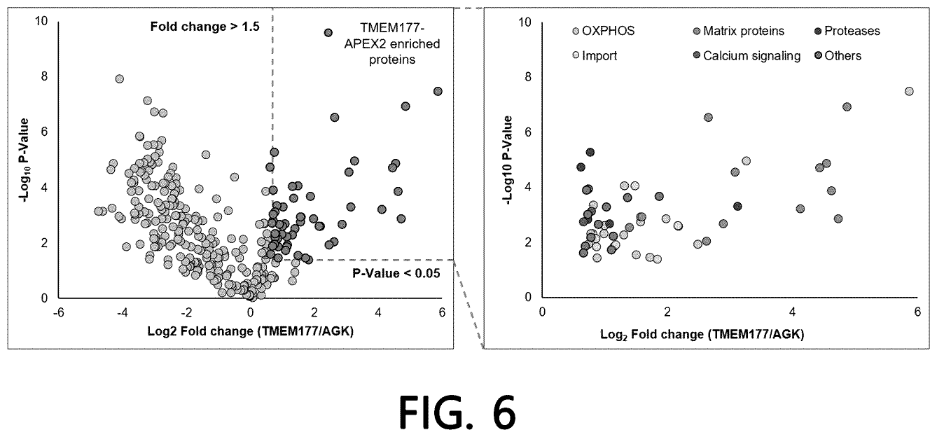

As a result, as shown in FIG. 6, 56 crista lumen proteins, including TMEM177, were identified, and these 56 proteins were mainly found to be oxidative phosphorylation complexes, proteases, and mitochondrial matrix proteins.

Mitochondrial matrix proteins included mainly oligomerized proteins such as HSPD1, HSPE1, ATP5B and the like.

### Example 5. Verification of iSPOT-ID Analysis Results Using TMEM177

In order to prove that oligomerized proteins exist in the crista lumen based on the results obtained in Example 4, BamHI and NotI restriction enzymes were used in the pCDNA5 vector including the MTS sequence to be expressed by fusing an myc epitope tag and a mitochondrial targeting sequence (MTS) to dsRed, which forms a tetramer, or mGFP, which is monomerized, to perform cloning.

SEQ ID NO: 5:
MTS-myc-dsRed sequence
ATGCTGGCCACCCGCGTGTTCAGCCTGGTGGGCAAGCGCGCCATC
AGCACCAGCGTGTGCGTGCGCGCCCACaaggatccagaacaaaaa
ctcatctcagaagaggatctgGCCTCCTCCGAGGACGTCATCAAG
GAGTTCATGCGCTTCAAGGTGCGCATGGAGGGCTCCGTGAACGGC
CACGAGTTCGAGATCGAGGGCGAGGGCGAGGGCCGCCCCTACGAG
GGCACCCAGACCGCCAAGCTGAAGGTGACCAAGGGCGGCCCCCTG
CCCTTCGCCTGGGACATCCTGTCCCCCCAGTTCCAGTACGGCTCC
AAGGTGTACGTGAAGCACCCCGCCGACATCCCCGACTACAAGAAG
CTGTCCTTCCCCGAGGGCTTCAAGTGGGAGCGCGTGATGAACTTC
GAGGACGGCGGCGTGGTGACCGTGACCCAGGACTCCTCCCTGCAG
GACGGCTCCTTCATCTACAAGGTGAAGTTCATCGGCGTGAACTTC
CCCTCCGACGGCCCCGTAATGCAGAAGAAGACTATGGGCTGGGAG
GCCTCCACCGAGCGCCTGTACCCCCGCGACGGCGTGCTGAAGGGC
GAGATCCACAAGGCCCTGAAGCTGAAGGACGGCGGCCACTACCTG
GTGGAGTTCAAGTCCATCTACATGGCCAAGAAGCCCGTGCAGCTG
CCCGGCTACTACTACGTGGACTCCAAGCTGGACATCACCTCCCAC
AACGAGGACTACACCATCGTGGAGCAGTACGAGCGCGCCGAGGGC
CGCCACCACCTGTTCCTGTAATGA
SEQ ID NO: 6:
MTS-myc-mGFP sequence
ATGCTGGCCACCCGCGTGTTCAGCCTGGTGGGCAAGCGCGCCATC
AGCACCAGCGTGTGCGTGCGCGCCCACaaggatccagaacaaaaa
ctcatctcagaagaggatctgGTGAGCAAGGGCGAGGAGCTGTTC
ACCGGGGTGGTGCCCATCCTGGTCGAGCTGGACGGCGACGTAAAC
GGCCACAAGTTCAGCGTGTCCGGCGAGGGCGAGGGCGATGCCACC
TACGGCAAGCTGACCCTGAAGTTCATCTGCACCACCGGCAAGCTG
CCCGTGCCCTGGCCCACCCTCGTGACCACCCTGACCTACGGCGTG
CAGTGCTTCAGCCGCTACCCCGACCACATGAAGCAGCACGACTTC
TTCAAGTCCGCCATGCCCGAAGGCTACGTCCAGGAGCGCACCATC
TTCTTCAAGGACGACGGCAACTACAAGACCCGCGCCGAGGTGAAG
TTCGAGGGCGACACCCTGGTGAACCGCATCGAGCTGAAGGGCATC
GACTTCAAGGAGGACGGCAACATCCTGGGGCACAAGCTGGAGTAC
AACTACAACAGCCACAACGTCTATATCATGGCCGACAAGCAGAAG
AACGGCATCAAGGTGAACTTCAAGATCCGCCACAACATCGAGGAC
GGCAGCGTGCAGCTCGCCGACCACTACCAGCAGAACACCCCCATC
GGCGACGGCCCCGTGCTGCTGCCCGACAACCACTACCTGAGCACC
CAGTCCAAGCTGAGCAAAGACCCCAACGAGAAGCGCGATCACATG
GTCCTGCTGGAGTTCGTGACCGCCGCCGGGATCACTCTCGGCATG
GACGAGCTGTACAAG

The vector was respectively expressed in TMEM177-V5-APEX2 and AGK-V5-APEX2 cell lines through polyethylenimine (PEI, Polysciences) transfection, and then labeled with LDBP. The labeling method was the same as in Example 2. Meanwhile, CHCHD3, which is an intermembrane space protein, was used as a control.

After the protein was dissolved with RIPA (Elpisbio) lysis buffer supplemented with a protease inhibitor cocktail (Thermofisher), LDBP-labeled proteins were isolated by using immunoprecipitation through streptavidin beads. After performing electrophoresis of the isolated proteins and the proteins before isolation at 200V for 1 hour using a 10% SDS-PAGE gel, the proteins were transferred to a nitrocellulose membrane (Pall Incorporation) by using a Tris-glycine-based buffer. Afterwards, the gel was removed from the membrane and blocked at room temperature for 1 hour using 1×TBST (TBS+0.1% tween 20, Sigma Aldrich) containing 2% skim milk powder. Additionally, the anti-myc antibody (Santa Cruz Biotechnology) and anti-CHCHD3 antibody (Sigma Aldrich), which are primary antibodies, were each diluted at 1:2000 in 1×TBST containing 2% skim milk powder and reacted at 4° C. for 12 hours. Residual primary antibodies were washed four times for 5 minutes each using 1×TBST, and secondary antibodies (anti-rabbit-HRP, cell signaling) were diluted at 1:3000 in 1×TBST containing 2% skim milk powder and reacted at room temperature for 1 hour. In order to confirm the protein labeled with LDBP, streptavidin-HRP was treated at room temperature for 30 minutes on a membrane that was not treated with any primary antibody. Likewise, after washing with 1×TBST, the results for each antibody were confirmed trough the ECL solution (Biorad).

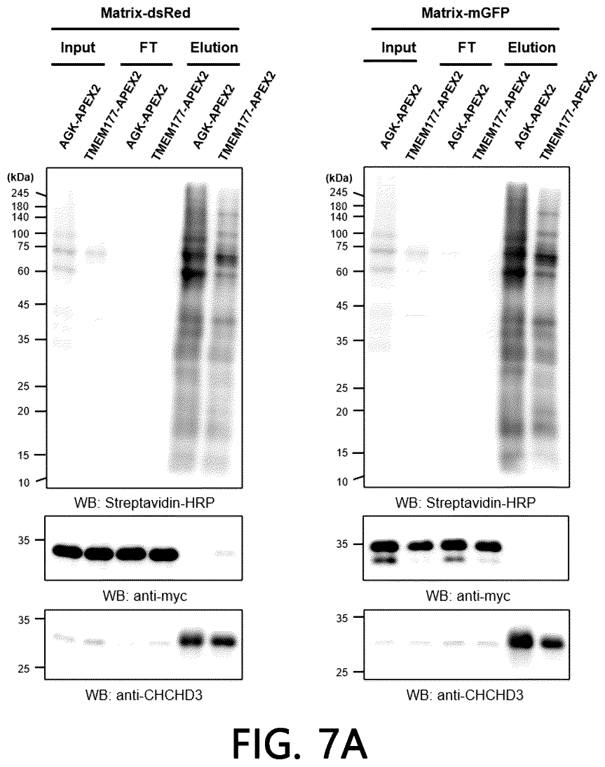

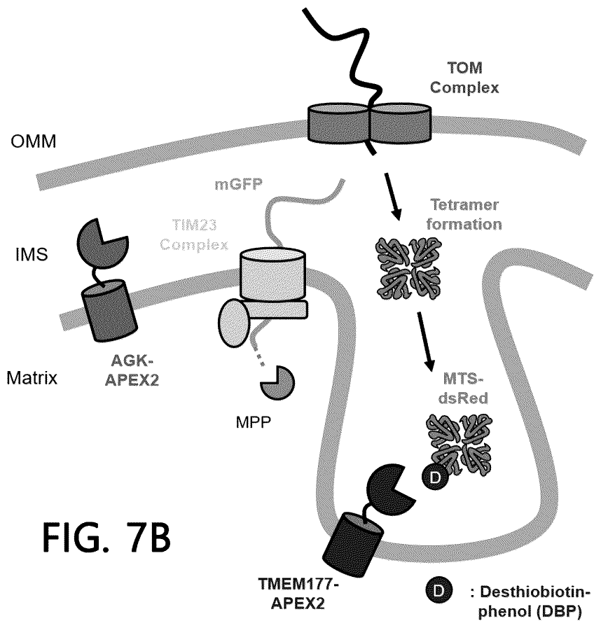

As a result, as shown in FIG. 7a, it was confirmed that dsRed was located in the crista lumen through Western blotting with an anti-myc antibody, and there was no difference in the CHCHD3 antibody used as a control. From the above results, it could be predicted that when the matrix protein is oligomerized or aggregated, it moves into the crista lumen, as shown in FIG. 7b.

As the specific parts of the present invention have been described in detail above, it is clear to those skilled in the art that these specific descriptions are merely preferred exemplary embodiments and do not limit the scope of the present invention. Accordingly, the substantial scope of the present invention will be defined by the appended claims and their equivalents.
IMM: Inner mitochondrial membrane
OMM: Outer mitochondrial membrane
IMS: Mitochondrial intermembrane space
ICS: Intracristal space
OCS: Outer ICS
BP: Biotin-phenol
HBP: Heavy Biotin-phenol
DBP: Desthiobiotin-phenol
HDBP: Heavy Desthiobiotin-phenol

### National Research and Development Project that Supported this Invention

[Task Unique Number]1465033955
[Task Number] HU20C0326010021
[Name of Ministry] Ministry of Health and Welfare
[Name of Task Management (Professional) Organization] Korea Health Industry Development Institute
[Research Project Name] Dementia Overcoming Research and Development Project (R&D)
[Research Task Name] Identification of pathogenesis of Alzheimer's disease based on TREM2 proteome analysis of MAM using Spot-TurboID
[Contribution rate]1/2
[Name of Task Performance Organization] Seoul National University Industry-Academic Cooperation Foundation
[Research Period] Jun. 1, 2021 to Mar. 31, 2022
[Task Unique Number]1711155246
[Task Number]2022R1A2B5B03001658
[Name of Ministry] Ministry of Science and ICT
[Name of Task Management (Professional) Organization] National Research Foundation of Korea
[Research Project Name] Individual Basic Research (Ministry of Science and ICT)
[Research Task Name] Identification of new retrograde signaling pathway in mitochondria
[Contribution rate]1/2
[Name of Task Performance Organization] Seoul National University
[Research Period] Mar. 1, 2022 to Feb. 28, 2023

1. A phenolic compound represented by Chemical Formula 1 or Chemical Formula 2 below:

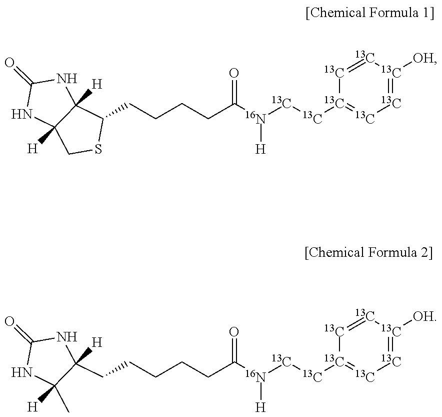

2. The phenolic compound of claim 1, wherein the phenolic compound is a probe for proximity molecular labeling.

3. A method for preparing the compound of claim 1, comprising the following steps:
(a) adding pyridoxal-5-phosphate to ¹³C₉ tyrosine or ¹³C₉ ¹⁵N tyrosine to react with tyrosine decarboxylase to respectively produce ¹³C₈ tyramine or ¹³C₈ ¹⁵N tyramine; and
(b) respectively reacting the produced ¹³C₈ tyramine or ¹³C₈ ¹⁵N tyramine with d-Biotinyl-NHS ester or d-Desthiobiotinyl-NHS ester to respectively produce a compound of Chemical Formula 1 or Chemical Formula 2.

4. A composition for labeling a protein or peptide, comprising the compound of claim 1.

5. The composition of claim 4, wherein the composition for labeling a protein or peptide comprises:
(i) biotin phenol and heavy biotin phenol represented by Chemical Formula 1; or
(ii) desthiobiotin phenol and heavy desthiobiotin phenol represented by Chemical Formula 2.

6. A method for measuring a protein ratio in adjacent spaces within cells using the compound of claim 1, comprising the following steps:
(a) expressing (i) a first fusion protein in which an APEX family enzyme is fused with a membrane protein located in a first membrane that separates a first space and a second space within a cell, or (ii) a first fusion protein in which an APEX family enzyme is fused with a protein located in the intermembrane space between a first membrane that separates a first space and a second space and a second membrane that separates a second space and a third space within a cell;
(b) expressing a second fusion protein in which a membrane protein of the second membrane and an APEX family enzyme are fused to the second membrane that separates a second space and a third space within a cell, in a cell that is homologous to the cell expressing the first fusion protein;
(c) treating the cell expressing the first fusion protein with biotin phenol or desthiobiotin phenol, and treating the cell expressing the second fusion protein with heavy biotin phenol represented by Chemical Formula 1 of claim 1 or heavy desthiobiotin phenol represented by Chemical Formula 2 of claim 1;
(d) lysing the cell expressing the first fusion protein and the cell expressing the second fusion protein to separate proteins from each cell; and
(e) mixing the proteins separated from the two cells and measuring the ratio of the biotin- or desthiobiotin-labeled protein and the heavy biotin- or heavy desthiobiotin-labeled protein.

7. A method for determining the intracellular location of a protein using the compound of claim 1, comprising the following steps:
(a) expressing (i) a first fusion protein in which an APEX family enzyme is fused with a membrane protein located in a first membrane that separates a first space and a second space within a cell, or (ii) a first fusion protein in which an APEX family enzyme is fused with a protein located in the intermembrane space between a first membrane that separates a first space and a second space and a second membrane that separates a second space and a third space within a cell;
(b) expressing a second fusion protein in which a membrane protein of the second membrane and an APEX family enzyme are fused to the second membrane that separates a second space and a third space within a cell, in a cell that is homologous to the cell expressing the first fusion protein;
(c) treating the cell expressing the first fusion protein with biotin phenol or desthiobiotin phenol, and treating the cell expressing the second fusion protein with heavy biotin phenol represented by Chemical Formula 1 of claim 1 or heavy desthiobiotin phenol represented by Chemical Formula 2 of claim 1;
(d) lysing the cell expressing the first fusion protein and the cell expressing the second fusion protein to separate proteins from each cell;
(e) mixing the proteins separated from the two cells and measuring the ratio of the biotin- or desthiobiotin-labeled protein and the heavy biotin- or heavy desthiobiotin-labeled protein; and
(f) determining whether the protein is located in the first space, second space or third space within the cell from the ratio of the labeled proteins.

8. The method of claim 7, wherein in step (e), the proteins that are separated from the two cells are mixed at a weight ratio of 1:1.

9. The method of claim 8, wherein the first membrane is an outer mitochondrial membrane (OMM), and the second membrane is an inner mitochondrial membrane (IMM).

10. The method of claim 9, wherein when the first fusion protein is a fusion protein in which a membrane protein located in the first membrane that separates the first space and the second space within the cell is fused with an APEX family enzyme, the protein labeled only by the second fusion protein is identified as a crista lumen protein.

11. The method of claim 8, wherein the first fusion protein is present in the outer space of the crista lumen (Outer ICS), and the second fusion protein is present in the intracristal space (ICS).

12. The method of claim 11, wherein when the ratio of labeling by the second fusion protein to the ratio of labeling by the first fusion protein is 1.5 or more and the P-value is 0.05 or less, the protein is identified as a crista lumen protein.

13. A method for measuring an intercellular protein ratio using the compound of claim 1, comprising the following steps:
(a) expressing a fusion protein in which an intracellular protein or membrane protein of a first cell is fused with an APEX family enzyme;
(b) expressing a fusion protein in which an intracellular protein or membrane protein of a second cell is fused with an APEX family enzyme;
(c) treating the first cell with biotin phenol or desthiobiotin phenol, and treating the second cell with heavy biotin phenol represented by Chemical Formula 1 of claim 1 or heavy desthiobiotin phenol represented by Chemical Formula 2 of claim 1;
(d) lysing the first cell and the second cell to separate proteins from each cell; and
(e) mixing the proteins separated from the two cells and measuring the intercellular protein ratio by measuring the ratio of the biotin- or desthiobiotin-labeled protein and the heavy biotin- or heavy desthiobiotin-labeled protein.

14. The method of claim 13, wherein the intracellular protein or membrane protein of the first cell and the intracellular protein or membrane protein of the second cell are homologous proteins.

15. The method of claim 13, wherein (i) the first cell is a normal cell and the second cell is a diseased cell;
(ii) the first cell is a diseased cell, and the second cell is a diseased cell that is treated with a drug; or
(iii) the first cell is a cell derived from one tissue in vivo, and the second cell is a cell derived from a different tissue in vivo.


SEQUENCE LISTING (6 sequences)

SEQ ID NO: 1 - DNA, 2532 bp, synthetic construct
  source 1..2532: mol_type = other DNA
atgtctactgaacggacttcttggacaagcctgtccaccattcagaaaatagccctgggccttgggatcccagccagtgcaacagttgcctatatcctataccgcaggtatagggaaagcagagaagagcggctgacatttgttggggaagatgacattgagatagagatgcgggttccccaggaggctgtgaaactcatcattggccggcaaggagccaatattaaacagctgcggaaacagacaggtgctcggattgatgtggacacagaggatgtaggcgatgagcgagtgctgcttatcagtggttttcctgttcaggtgtgcaaggccaaagcagcaatccatcagatcctgacagagaataccccagtgtctgagcagctttcagttccccagagatctgtgggcagaatcatagggagaggcggcgagacaattcgttctatctgtaaggcatctggagccaaaattacctgtgacaaagaatcagaagggacattactactatcaagacttataaaaatctcaggaacacagaaggaagtggcagcagccaagcatttgatactggagaaagtttcagaagatgaagaacttcggaagagaattgctcattctgcagaaaccagggtcccacgcaaacagccaatcagtgtgagaagagaagacatgacagagccaggtggagctggagagccagcattatggaaaaacaccagttctagcatggagccgactgcacccctggtgactcctccacccaaaggaggaggcgacatggctgtggtagtgtcaaaggaaggttcctgggagaaacctagtgatgacagctttcagaagtctgaagcccaggccatcccagagatgcccatgtttgaaatccccagtcctgacttcagttttcatgctgatgagtacctagaagtctacgtttctgcttctgagcaccctaaccacttctggatccagatcgttggctcccgcagcctgcaattggataagcttgtcaatgagatgacccagcactatgagaatagtgtgcctgaagacttgactgtgcatgtaggagacattgtagcagcacctttacctacaaatggttcctggtatcgagcccgggtcctcggcaccttggagaatgggaacttggacctctattttgttgactttggagataatggagattgcccactgaaggacctcagggctctcaggagtgacttcctaagccttccatttcaagcaatagaatgtagtctggcacggattgctccctcaggtgaccagtgggaagaggaagctttggatgagtttgatagactcactcattgtgctgactggaagcctctggtagccaagatctctagctatgtccagactgggatctcaacttggccaaagatctacttatatgatactagcaatgggaagaaacttgatattgggctagaattagtacacaaaggatacgcaattgagcttcctgaagacatagaagaaaacagagctgtcccagacatgttgaaggacatggccacagaaacagatgcctctctcagcacgttgctcactgagaccaaaaagagctctggagagataacacataccctgtcctgcctcagcttatcagaagctgcttccatgtctggtgatgataaccttgaagatgactacttactcaaggatccgctagcaggcaagcccatccccaaccccctgctgggcctggacagcaccggaaagtcttacccaactgtgagtgctgattaccaggacgccgttgagaaggcgaagaagaagctcagaggcttcatcgctgagaagagatgcgctcctctaatgctccgtttggcattccactctgctggaacctttgacaagggcacgaagaccggtggacccttcggaaccatcaagcaccctgccgaactggctcacagcgctaacaacggtcttgacatcgctgttaggcttttggagccactcaaggcggagttccctattttgagctacgccgatttctaccagttggctggcgttgttgccgttgaggtcacgggtggacctaaggttccattccaccctggaagagaggacaagcctgagccaccaccagagggtcgcttgcccgatcccactaagggttctgaccatttgagagatgtgtttggcaaagctatggggcttactgaccaagatatcgttgctctatctgggggtcacactattggagctgcacacaaggagcgttctggatttgagggtccctggacctctaatcctcttattttcgacaactcatacttcacggagttgttgagtggtgagaaggaaggtctccttcagctaccttctgacaaggctcttttgtctgaccctgtattccgccctctcgttgataaatatgcagcggacgaagatgccttctttgctgattacgctgaggctcaccaaaagctttccgagcttgggtttgctgatgccggcctgaacgacatcttcgaggcccagaagatcgagtggcacgag

SEQ ID NO: 2 - DNA, 1701 bp, synthetic construct
  source 1..1701: mol_type = other DNA
atggcgatgctggtcctagtacccggacgagttatgcggcctctgggtggccaactttggcgcttcttgcctcgcggactcgagttttggggcccagccgaggggactgcgagagtcttgctgaggcagttctgcgcgcggcaagcggaggcgtggcgtgcctcggggcgccctggctattgcctgggaacccggcccctcagcactgcgaggccgccacccccgtggtcgcagaagggccccggagactccacgcgcccctcgaagcccgggcctgtttcctggaagtctttagcaatcacatttgctattggaggagctttactggctggaatgaagcacgtcaagaaagaaaaggcagagaagttagagaaggaacggcagcgacacatcggcaagcctttacttgggggaccgttttccctcacaactcatactggggagcgtaaaactgacaaggactacttgggtcagtggttattgatttattttggcttcactcattgccctgatgtctgtccagaagaactagaaaagatgattcaagtcgtggatgaaatagatagcattacaactctgccagatctaactccacttttcatcagcattgacccagagagggacacaaaagaagccatcgcaaattatgtgaaagaattttctcccaaactggttggcttgactggcacgagagaagaggtcgatcaagtggccagagcatacagagtgtattacagccctggccccaaggacgaagatgaagactacatagtggatcacacaataataatgtacttgattggaccagatggtgagtttctagattattttggccagaacaagaggaagggagaaatagctgcttcaattgccacacacatgaggccatacagaaaaaagagcaaggatccaggcaagcccatccccaaccccctgctgggcctggacagcaccggaaagtcttacccaactgtgagtgctgattaccaggacgccgttgagaaggcgaagaagaagctcagaggcttcatcgctgagaagagatgcgctcctctaatgctccgtttggcattccactctgctggaacctttgacaagggcacgaagaccggtggacccttcggaaccatcaagcaccctgccgaactggctcacagcgctaacaacggtcttgacatcgctgttaggcttttggagccactcaaggcggagttccctattttgagctacgccgatttctaccagttggctggcgttgttgccgttgaggtcacgggtggacctaaggttccattccaccctggaagagaggacaagcctgagccaccaccagagggtcgcttgcccgatcccactaagggttctgaccatttgagagatgtgtttggcaaagctatggggcttactgaccaagatatcgttgctctatctgggggtcacactattggagctgcacacaaggagcgttctggatttgagggtccctggacctctaatcctcttattttcgacaactcatacttcacggagttgttgagtggtgagaaggaaggtctccttcagctaccttctgacaaggctcttttgtctgaccctgtattccgccctctcgttgataaatatgcagcggacgaagatgccttctttgctgattacgctgaggctcaccaaaagctttccgagcttgggtttgctgatgcc

SEQ ID NO: 3 - DNA, 933 bp, Homo sapiens
  source 1..933: mol_type = other DNA
atggcaggtcccctgtggcggaccgcagcatttgtgcagagacacaggacaggcctcttggtgggttcctgtgcaggcctgtttggagttccagtctcgtaccacctcttcccggatcccgtggtccaatggctctaccagtactggcctcagggccagccagctccgctccctccacagctgcagagcctcttccaagaggtgctacaggacataggtgttccttcaggccattgctacaagcccttcaccaccttcaccttccagcctgtgagtgcaggcttcccaagactccctgctggggctgtggtgggcatccctgccagtttcttgggagacctagtgatcaacactaaccatcccgtggtcatacatgggcatacagtggactggcggagcccagcaggcgcccggctgagagcttccctgaccttgtcccgtgaagcccagaagttcgccttggccagggaagtggtgtacctggaaagcagtaccactgccgtgcacgccctgctggccccagcttgcctggcagggacctgggcactgggcgtgggtgccaagtacaccctggggctccatgcaggccccatgaatttacgggctgccttcagcttggtggcagcagtggcaggctttgtggcctacgccttctcccaggattctctcactcatgccgtggagtcctggctggaccgccgcacggcctccctctctgcagcctatgcctgtggtggagtggagttctatgagaagcttctgtcgggcaacctggccctgcgcagtctcttgggcaaagagggggagaagctgtatacacccagcgggaacatcgtccccagacacttgttccgaatcaaacatttaccctacaccacccgccgggactctgtgctgcagatgtggagggggatgctcaatccgggccgctcc

SEQ ID NO: 4 - DNA, 1266 bp, Homo sapiens
  source 1..1266: mol_type = other DNA
atgacggtgttctttaaaacgcttcgaaatcactggaagaaaactacagctgggctctgcctgctgacctggggaggccattggctctatggaaaacactgtgataacctcctaaggagagcagcctgtcaagaagctcaggtgtttggcaatcaactcattcctcccaatgcacaagtgaagaaggccactgtttttctcaatcctgcagcttgcaaaggaaaagccaggactctatttgaaaaaaatgctgccccgattttacatttatctggcatggatgtgactattgttaagacagattatgagggacaagccaagaaactcctggaactgatggaaaacacggatgtgatcattgttgcaggaggagatgggacactgcaggaggttgttactggtgttcttcgacgaacagatgaggctaccttcagtaagattcccattggatttatcccactgggagagaccagtagtttgagtcataccctctttgccgaaagtggaaacaaagtccaacatattactgatgccacacttgccattgtgaaaggagagacagttccacttgatgtcttgcagatcaagggtgaaaaggaacagcctgtatttgcaatgaccggccttcgatggggatctttcagagatgctggcgtcaaagttagcaagtactggtatcttgggcctctaaaaatcaaagcagcccactttttcagcactcttaaggagtggcctcagactcatcaagcctctatctcatacacgggacctacagagagacctcccaatgaaccagaggagacccctgtacaaaggccttctttgtacaggagaatattacgaaggcttgcgtcctactgggcacaaccacaggatgccctttcccaagaggtgagcccggaggtctggaaagatgtgcagctgtccaccattgaactgtccatcacaacacggaataatcagcttgacccgacaagcaaagaagattttctgaatatctgcattgaacctgacaccatcagcaaaggagactttataactataggaagtcgaaaggtgagaaaccccaagctgcacgtggagggcacggagtgtctccaagccagccagtgcactttgcttatcccggagggagcagggggctcttttagcattgacagtgaggagtatgaagcgatgcctgtggaggtgaaactgctccccaggaagctgcagttcttctgtgatcctaggaagagagaacagatgctcacaagccccacccag

SEQ ID NO: 5 - DNA, 789 bp, synthetic construct
  source 1..789: mol_type = other DNA
atgctggccacccgcgtgttcagcctggtgggcaagcgcgccatcagcaccagcgtgtgcgtgcgcgcccacaaggatccagaacaaaaactcatctcagaagaggatctggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtaatga

SEQ ID NO: 6 - DNA, 825 bp, synthetic construct
  source 1..825: mol_type = other DNA
atgctggccacccgcgtgttcagcctggtgggcaagcgcgccatcagcaccagcgtgtgcgtgcgcgcccacaaggatccagaacaaaaactcatctcagaagaggatctggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaag